(19) United States
(12) Patent Application Publication
(10) Pub. No.: US 2022/0370384 A1
(43) Pub. Date: Nov. 24, 2022

(54) COMBINATIONS OF MEK INHIBITORS WITH CAP-DEPENDENT ENDONUCLEASE INHIBITORS

(71) Applicant: Atriva Therapeutics GmbH, Tuebingen (DE)

(72) Inventors: Oliver Planz, Dettingen an der Erms (DE); Hazem Ewess, Tuebingen (DE)

(21) Appl. No.: 17/638,440

(22) PCT Filed: Aug. 27, 2020
(86) PCT No.: PCT/EP2020/073934

§ 371 Date: Feb. 25, 2022

(30) Foreign Application Priority Data
Aug. 27, 2019 (LU) LU101372

(51) Int. Cl. A61K 31/166; A61K 31/5383; A61K 45/06; A61P 31/12

(52) U.S. Cl. CPC A61K 31/166; A61K 31/5383; A61K 45/06; A61P 31/12

(57) ABSTRACT

The present invention relates to MEK inhibitors that are capable of displaying one or more beneficial therapeutic effects. The MEK inhibitors can be used in the prevention and/or treatment of viral infection. MEK inhibitors in combination with a cap-dependent endonuclease inhibitor are capable of displaying one or more beneficial therapeutic effects in the treatment of viral diseases.

### FIELD OF THE INVENTION

The present invention relates to the combination of MEK inhibitors that are capable of displaying one or more beneficial therapeutic effects with cap-dependent endonuclease (CEN) inhibitors such as Baloxavir marboxil. The MEK inhibitors can be used together with the cap-dependent endonuclease inhibitors in the prevention and/or treatment of viral infection. MEK inhibitors in combination with cap-dependent endonuclease inhibitors are capable of displaying one or more improved beneficial therapeutic effects in the treatment of viral diseases.

### BACKGROUND OF THE INVENTION

Infections with RNA or DNA viruses are a significant threat for the health of man and animal. For instance, infections with influenza viruses do still belong to the big epidemics of mankind and cause year for year a big number of casualties. In terms of the national economies, they are an immense cost factor, for instance due to unfitness for work. Infections with the Borna disease virus (BDV), which mainly affects horses and sheep, but which has also been isolated for humans and is connected to neurological diseases, equally have an enormous economic importance.

The problem of controlling in particular RNA viruses is the adaptability of the viruses caused by a high fault rate of the viral polymerases, which makes the production of suitable vaccines as well as the development of antiviral substances very difficult. Furthermore it has been found that while the application of antiviral substances immediately directed against the functions of the virus show a good antiviral effect at the beginning of the treatment, these quickly lead to the selection of resistant variants based on mutation. An example is the anti-influenza agent amantadine and its derivatives directed against a transmembrane protein of the virus. Within a short time after the application, resistant variants of the virus are generated. Other examples are the new therapeutics for influenza infections inhibiting the influenza-viral surface protein neuraminidase, such as Relenza. In patients, Relenza-resistant variants have already been found (Gubareva et al., J Infect Dis 178, 1257-1262, 1998).

The drawback of prior art antiviral active substances is that they are either directed against a viral component and thus quickly lead to resistances (cf. amantadine), or act in a too broad and unspecific manner against cellular factors (for example methyl transferase inhibitors), and significant side effects are to be expected.

A new class of antivirals has recently been identified, the cap-dependent endonuclease inhibitors. These inhibitors target the cap-dependent endonuclease (CEN), which resides in the PA subunit of influenza virus polymerase and mediates the “cap-snatching” process during viral mRNA biosynthesis. S-033188, also called Baloxavir marboxil, is a potent, selective, small molecule inhibitor of CEN that has been approved by the FDA in October 2018 under the trade name Xofluza® for the treatment of influenza. However, first cases of virus resistance have already been reported for Baloxavir marboxil and are expected for other CEN inhibitors.

Because of the very small genome and thus limited coding capacity for functions being necessary for the replication, all viruses are dependent to a high degree on functions of their host cells. By exertion of influence on such cellular functions necessary for viral replication, it is possible to negatively affect the virus replication in the infected cell. In this scenario, there is no possibility for the virus to replace the lacking cellular function by adaptation, in particular by mutations, in order to thus escape from the selection pressure. This could already be shown for the influenza A virus with relatively unspecific inhibitors against cellular kinases and methyl transferases (Scholtissek and Müller, Arch Virol 119, 111-118, 1991).

It is known in the art that cells have a multitude of signal transmission paths, by means of which signals acting on the cells are transmitted into the cell nucleus. Thereby the cell is capable to react to external stimuli and to react by cell proliferation, cell activation, differentiation, or controlled cell death. It is common to these signal transmission paths that they contain at least one kinase activating by phosphorylation at least one protein subsequently transmitting a signal. When observing the cellular processes induced after virus infections, it is found that a multitude of DNA and RNA viruses preferably activate in the infected host cell a defined signal transmission path, the so-called Raf/MEK/ERK kinase signal transmission path (Benn et al., J Virol 70, 4978-4985, 1996; Bruder and Kovesdi, J Virol 71, 398-404, 1997; Popik and Pitha, Virology 252, 210-217, 1998; Rodems and Spector, J Virol 72, 9173-9180, 1998). This signal transmission path is one of the most important signal transmission paths in a cell and plays a significant role in proliferation and differentiation processes. Growth factor-induced signals are transmitted by successive phosphorylation from the serine/threonine kinase Raf to the dual-specific kinase MEK (MAP kinase kinase/ERK kinase) and finally to the kinase ERK (extracellular signal regulated kinase). Whereas as a kinase substrate for Raf, only MEK is known, and the ERK isoforms were identified as the only substrates for MEK, ERK is able to phosphorylate a whole number of substrates. To these belong for instance transcription factors, whereby the cellular gene expression is directly influenced (Cohen, Trends in Cell Biol 7, 353-361, 1997; Robinson and Cobb, Curr. Opin. Cell Biol 9, 180-186, 1997; Treisman, Curr. Opin. Cell Biol 8, 205-215, 1996).

In view of the prior art, it is clear that there is the need of further compounds and compositions effective in the treatment of virus diseases in particular in diseases caused by influenza virus, in particular to avoid the formation of resistance.

In this regard, ongoing research on the usefulness of MEK inhibitors in the treatment of viral disease, in particular influenza, has revealed that this class of compounds avoids the disadvantages of the standard antiviral treatments as it is directed to cellular components of the host cells rather than towards the virus itself. For this reason, no resistance to MEK inhibitors has been observed. WO 2001/076570 provides for the concept of treating or preventing infections caused by (−)RNA viruses, in particular by influenza viruses by way of MEK inhibitors. WO 2014/056894 provides for specific MEK inhibitors, such as AZD-6244, AZD-8330, RDEA-119, GSK-1120212 (Trametinib), GDC-0973 (Cobimetinib), CI-1040, PD-0325901, RO-5126766, MSC1936369 (AS-703026) for use in the treatment or prevention of influenza virus infections. In WO 2015/173788 A1 MEK inhibitors are disclosed for use in a method of treating influenza virus and bacterial co-infections. In addition, WO 2019/076947 discloses a new MEK inhibitor, PD-0184264 (also known as ATR-002) for use in a method for the prophylaxis and/or treatment of a viral infection.

Nevertheless, there remains a need for the provision of further compositions and compounds for the treatment and prevention of viral infections.

### SUMMARY OF THE INVENTION

In the present invention, it was found that the use of a MEK inhibitor in the treatment or prevention of a viral infection in combination with a cap-dependent endonuclease inhibitor led to effective treatment of the viral infection. Specifically, a synergistic effect was seen when the MEK inhibitor PD-0184264 was administered together with Baloxavir marboxil.

In the context of the invention, the MEK inhibitor can be selected from the group consisting of CI-1040, PD-0184264, GSK-1120212, GDC-0973, PLX-4032, AZD6244, AZD8330, AS-703026, RDEA-119, RO-5126766, RO-4987655, PD-0325901, TAK-733, AS703026, PD98059 and PD184352 or pharmaceutically acceptable salt or metabolite thereof. In a preferred combination, the MEK inhibitor is CI-1040 or PD-0184264 and the cap-dependent endonuclease inhibitor is Baloxavir marboxil.

A preferred use is the treatment or prevention of a viral infection caused by a negative RNA strand virus, such as an influenza virus. The influenza virus can be influenza A virus or influenza B virus. In the context of the invention, the MEK inhibitor can be administered contemporaneously, previously or subsequently to the cap-dependent endonuclease inhibitor.

Also disclosed is a pharmaceutical composition comprising a MEK inhibitor or a pharmaceutically acceptable salt or metabolite thereof and a cap-dependent endonuclease inhibitor for use as a medicament, preferably for the treatment or prevention of viral disease such as influenza.

### FIGURES

FIG. 1 shows the antiviral activity of Oseltamivir and CI-1040 in comparison to a mock control against influenza virus H1N1 wildtype (white) and H1N1-H275Y (grey).

FIGS. 2a-b show the antiviral activity of Oseltamivir and Baloxavir marboxil in comparison to a mock control against influenza viruses H1N1 WT (white) and H11-PA-I38T (grey) as well as H3N2-WT (white) and H3N2-PA-I38T (grey).

FIGS. 3a-d show the synergistic effect between ATR002 and Baloxavir marboxil. Combinations of MEK inhibitor (ATR002) with Baloxavir marboxil (BLXM) were tested in 4×4 matrix (D) and all values normalized to Mock-infected control (DMSO). Contour and surface plots were generated by Combenefit upon processing data using three different synergy models: A) HAS; B) Bliss and C) Loewe. Areas with synergy scores above 25 are marked.

FIG. 4a shows the synergy/antagonism plotted as the Log (CI) on the y-axis versus the Fraction affected (Fa) on the x-axis.

FIG. 4b shows the Drug Reduction Index (DRI) of Baloxavir marboxil (BLXM) and ATR002 against influenza virus.

### DETAILED DESCRIPTION

The following description includes information that may be useful in understanding the present invention. It is not an admission that any of the information provided herein is prior art or relevant to the presently claimed inventions, or that any publication specifically or implicitly referenced is prior art.

“MEK inhibitors” as used herein inhibit the mitogenic signaling cascade Raf/MEK/ERK in cells or in a subject by inhibiting the MEK (mitogen-activated protein kinase kinase). This signaling cascade is hijacked by many viruses, in particular influenza viruses, to boost viral replication. Specific blockade of the Raf/MEK/ERK pathway at the bottleneck MEK therefore impairs growth of viruses, in particular influenza viruses. Additionally, MEK inhibitors show low toxicity and little adverse side effects in humans. There is also no tendency to induce viral resistance (Ludwig, 2009). A particularly preferred MEK inhibitor is PD-0184264 also known as ATR-002.

The MEK inhibitors preferably are selected from CI-1040, PD-0184264 GSK-1120212, GDC-0973, PLX-4032, AZD6244, AZD8330, AS-703026, RDEA-119, RO-5126766, RO-4987655, PD-0325901, TAK-733, AS703026, PD98059 and PD184352 or a pharmaceutically acceptable salt or a metabolite thereof. These MEK inhibitors are known in the art and, for example, described in Table 1 of Fremin and Meloche (2010), J. Hematol. Oncol. 11; 3:8. In the following, structural formulae of PD-0184264 and CI-1040 are shown for reference:

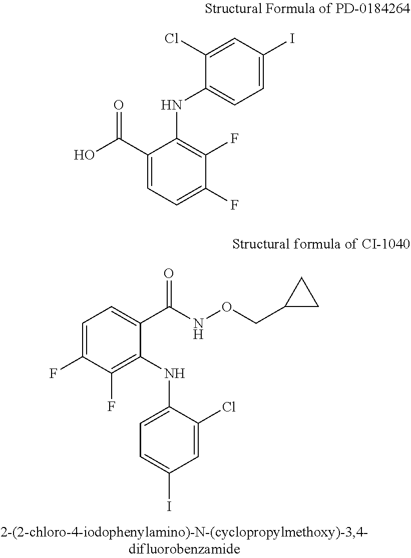 [SMILES: O=C(O)c1ccc(F)c(F)c1Nc1ccc(I)cc1Cl ; O=C(NOCC1CC1)c1ccc(F)c(F)c1Nc1ccc(I)cc1Cl]

A “metabolite” as used herein relates to an intermediate end product of metabolism of the MEK inhibitor, which arise during the degradation of the MEK inhibitor by the subject, e.g. in the liver. In a preferred embodiment, the MEK inhibitor is a metabolite of CI-1040, e.g., PD-0184264 is a metabolite of the MEK inhibitor CI-1040.

“Cap-dependent endonuclease (CEN) inhibitors” inhibit the CEN located in the N-terminal domain of the PA subunit of heterotrimeric RNA-dependent polymerase of influenza virus consisting of subunits PA, PB1 and PB2. This is essential for viral transcription and replication. In the process of ‘cap-snatching’, viral mRNA synthesis is initiated by PB2 binding to the cap structure of the host mRNA, followed by short-capped oligonucleotide cleavage by CEN. Intriguingly, CEN is well conserved among influenza virus strains and therefore considered to be an ideal anti-influenza virus drug target.

In a preferred embodiment, the CEN inhibitor is Baloxavir marboxil (formerly also denoted S-033188), a first-in-class antiviral drug for the treatment of influenza. After oral administration, Baloxavir marboxil may be metabolized to its active form (Baloxavir acid) that binds to CEN. The following structural formula shows Baloxavir marboxil:

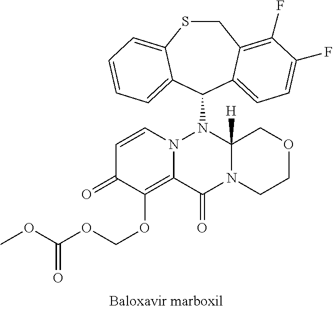 [SMILES: COC(=O)OCOc1c2n(ccc1=O)N([C@@H]1c3ccccc3SCc3c1ccc(F)c3F)[C@@H]1COCCN1C2=O]

For the purpose of the invention the active compound (MEK inhibitor and/or CEN inhibitor) as defined above also includes the pharmaceutically acceptable salt(s) thereof. The phrase “pharmaceutically or cosmetically acceptable salt(s)”, as used herein, means those salts of compounds of the invention that are safe and effective for the desired administration form. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylaminoethanol, histidine, procaine, etc.

As already outlined herein, Influenza viruses (IV) infection is still a public health concern worldwide. Currently, all available vaccines as well as antiviral drugs that target the virus itself are prone to resistance. It is proven that influenza viruses able to modulate and control cellular pathways involved in the viral life cycle like Raf/MEK/ERK signal pathway which the nuclear export of vRNPs is strongly dependent on the virus-induced activation. Along this line, the inventors demonstrated earlier the antiviral potential of MEK inhibitor PD0184264 (ATR002), the active metabolite of CI-1040 against influenza viruses over in vitro and in vivo levels (Example 1, see also WO 2019/076947). The newly licensed antiviral drug so-called Baloxavir marboxil (Xofluza), which was designed to inhibit the cap-dependent endonuclease protein, has demonstrated efficacy in a wide range of influenza viruses, including oseltamivir-resistant strains. However, the emergence of resistant variants against the newly licensed drug has already been reported.

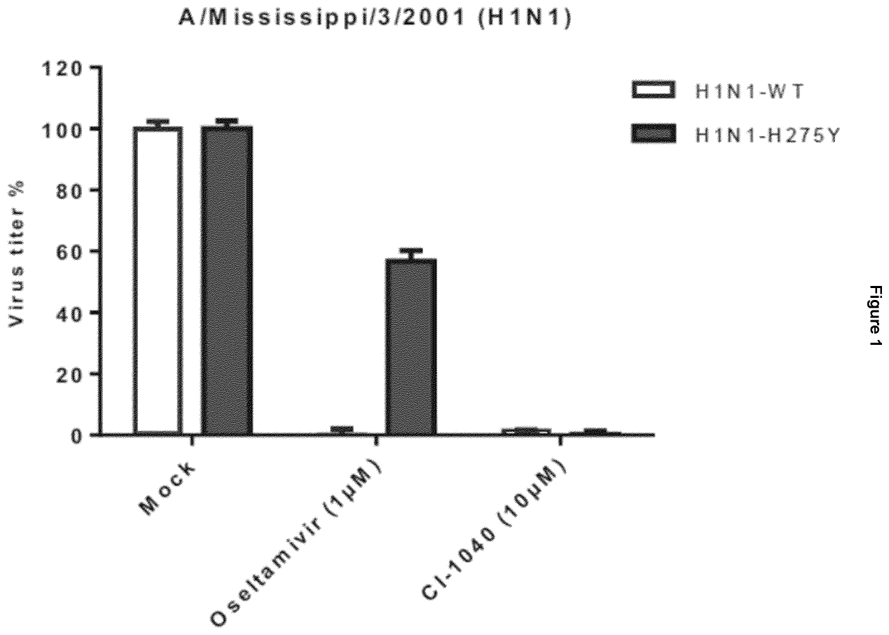

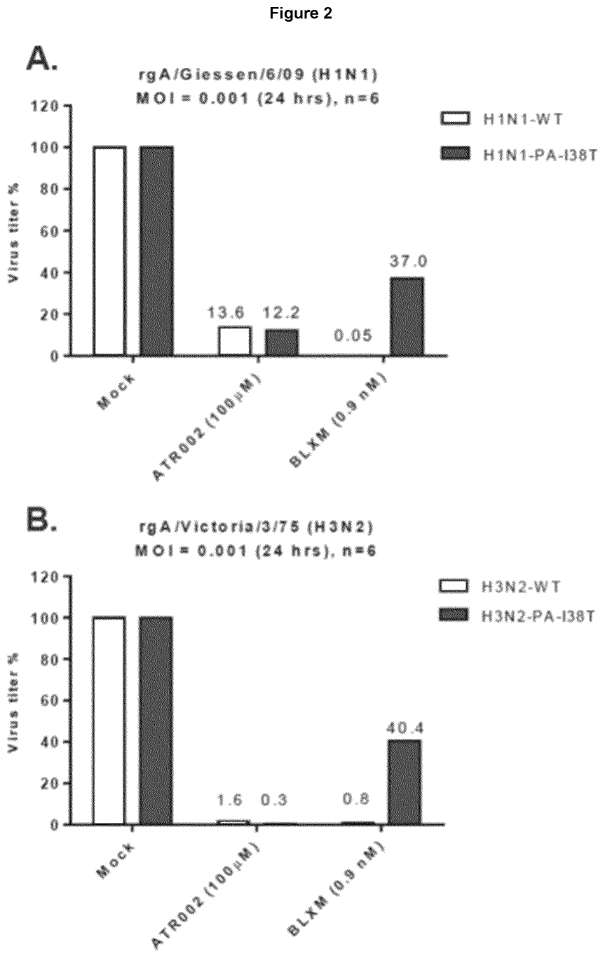

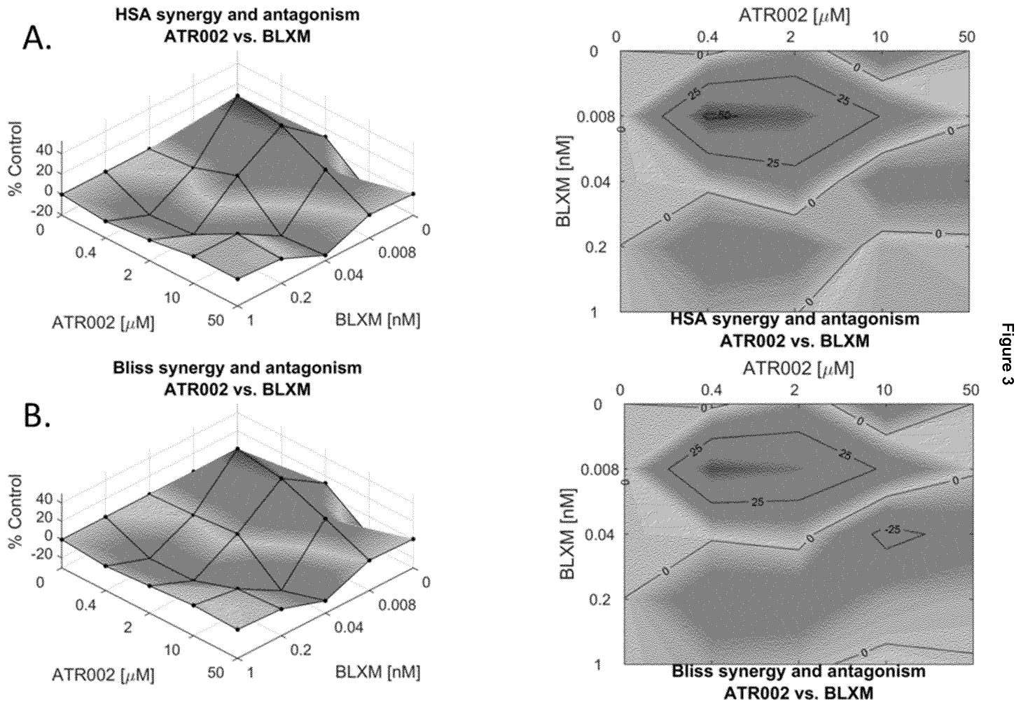

As shown in Example 1 and FIG. 1, both oseltamivir and CI-1040 are effective against wild type (wt) strain of Influenza A/Mississippi/3/2001 (H1N1). In contrast, while investigating the antiviral potential of both drugs against the mutant Influenza strain with the H275Y mutation in the neuraminidase (NA) gene, significant reduction in oseltamivir effectiveness was observed. CI-1040, in contrast, showed a comparable antiviral effect to that observed in the wild type strain. To further evaluate the potential antiviral activity of ATR002 (the active metabolite of CI-1040), the inventors compared the antiviral activity of ATR002 versus the newly licensed anti-influenza virus drug Baloxavir marboxil (BLXM) which is designed to inhibit the cap-dependent endonuclease protein. As shown in FIG. 2A, BLXM was found to be very potent against the wild type influenza rgA/Giessen/6/09 (H1N1-WT) with an approximate complete reduction of the viral titer while ATR002 activity was lower by 13%. Conversely, the BLXM activity was lower by 37% when investigated using the mutant strain rgA/Giessen/6/09 (H1N1)-PA-I38T but ATR002 showed the same effect as found in the wild type. Likewise, while investigating the antiviral activity using rgA/Victoria/3/75 (H3N2-WT) and rgA/Victoria/3/75 (H3N2-PA-I38T) (FIG. 2B), ATR002 revealed its potency against both variants, whereas, BLXM lost about 41% of its activity in the mutant variant.

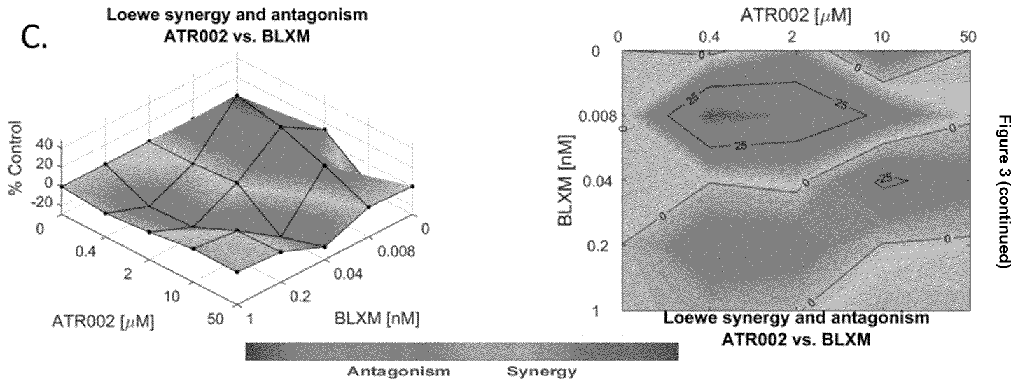

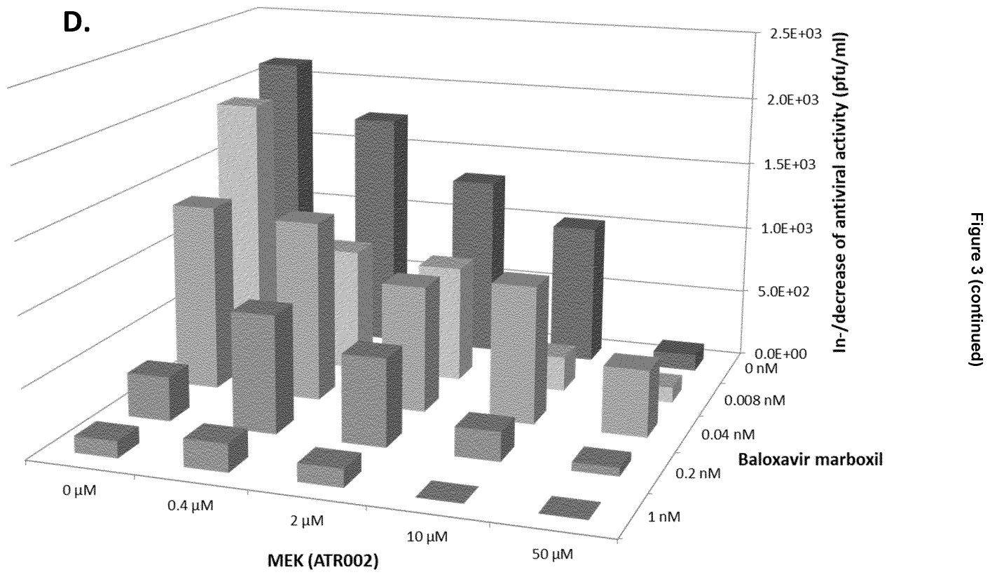

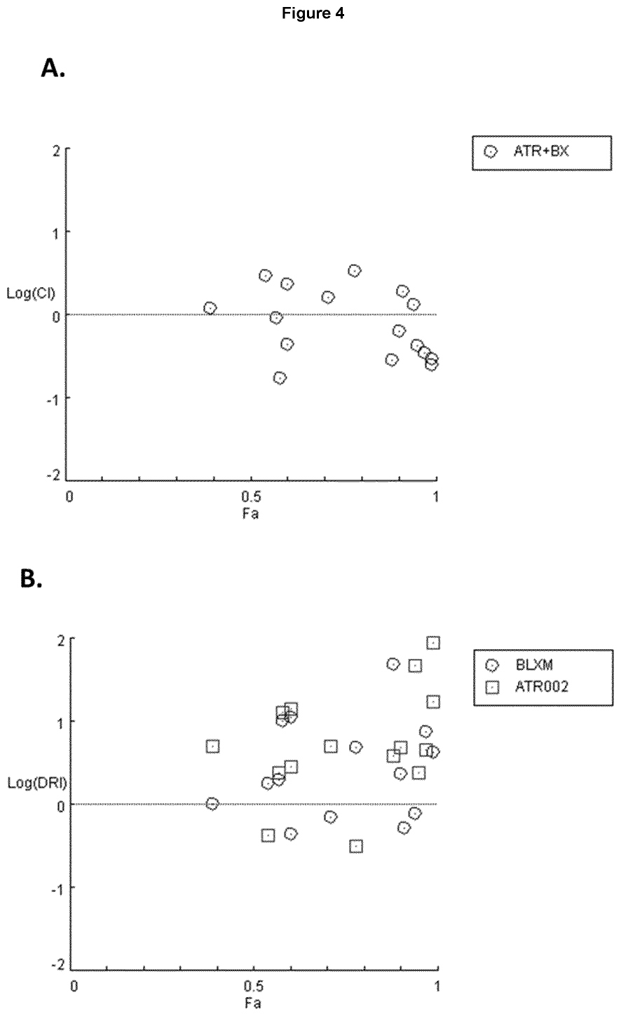

Given that both the recently licensed anti-influenza drug Baloxavir marboxil and the potential MEK inhibitor (ATR002) could be considered as a therapeutic option for influenza treatment, the inventors investigated in Example 2 whether the combination between these two drugs would augment the antiviral activity. There is a surprising increase in the antiviral activity at different concentrations of ATR002 (0.4, 2, and 10 μM) when combined with BLXM (0.008 and 0.04 nM) indicated by the reduction in viral titer compared to the individual treatment of each drug. Moreover, it can be inferred form Chou-Talalay model that the combination at lower concentrations of ATR002 and BLXM leads to a strong synergistic effect with low CI values (FIG. 4). These data were in agreement with the most widely used models (HAS, Bliss, and Loewe) which also revealed that the combinations at higher doses lead to stronger additive effect rather than synergistic effect (FIG. 3A-C).

Thus, the inventors surprisingly found that the combined administration of a MEK inhibitor and a CEN inhibitor creates unexpected synergies in preventing and/or treating viral diseases, in particular the combination of a MEK inhibitor and a CEN inhibitor led to a synergistic affect in inhibiting influenza A virus and/or B virus. Indeed, as shown herein, the MEK inhibitors CI-1040, PD-0184264 GSK-1120212, GDC-0973, PLX-4032, AZD6244, AZD8330, AS-703026, RDEA-119, RO-5126766, RO-4987655, PD-0325901, TAK-733, AS703026, PD98059 and PD184352 that are orally available and at least in a phase I clinical trial, some of them are even in a phase II clinical trial or even admitted for marketing, such as PLX-4032, against cancer, demonstrate antiviral activity, both against influenza A virus and/or influenza B virus, in combination with a CEN inhibitor, such as Baloxavir. Combination treatment increased the antiviral activity of Baloxavir significantly and resulted in a synergistic antiviral effect as determined by the HAS, Bliss and LOEWE methods described herein (FIG. 3). Taken together, the results demonstrate increased antiviral activity of Baloxavir after combination with MEK inhibitors, specifically PD-0184264 and CI-1040. These data are promising for further preclinical in vitro and in vivo investigations on the way to developing new antiviral regimens against influenza.

It hence has been found by the present inventors that the combination method of the invention is such that provide a synergy in the prevention and/or treatment of viral diseases, in particular in the prevention and/or treatment of an infection caused by a negative RNA strand virus more in particular viral diseases caused by influenza virus. Even more in particular in the prevention and/or treatment of in influenza A or B virus.

The above being said, the present invention relates to a MEK inhibitor for use in a method of prophylaxis and/or treatment of a viral infection in combination with a cap-dependent endonuclease inhibitor. The present invention further relates to a pharmaceutical composition comprising a MEK inhibitor or a pharmaceutically acceptable salt or metabolite thereof and a cap-dependent endonuclease inhibitor for use as a medicament. As shown in the examples, MEK inhibitors in combination with cap-dependent endonuclease inhibitors show a surprising synergistic antiviral effect.

The pharmaceutical composition of the invention may be administered in a synergistic amount.

“Synergy” or “synergistic effect” may be defined as an effect that is more than additive (Chou, 2006, Pharmacolog Reviews, 58: 621-681). Synergistic interactions amongst drug combinations are highly desirable and sought after since they can result in increased efficacy, decreased dosage, reduced side toxicity, and minimized development of resistance when used clinically (Chou, 2006). The two most popular methods for evaluating drug interactions in combination therapies are isobologram and combination index (CI) (Zhao et al., 2004, Clinical Cancer Res 10:7994-8004). Numerous studies in both the cancer therapy field and anti-viral therapy field, where drug combinations to counter the development of drug resistance and to minimize drug doses, use the CI index to evaluate synergy. CI is based on the approach of Chou and Talalay 1984 (Adv. Enzyme Regul. 22:27-55) and relies on the median effect principle and the multiple-drug effect equation. CI can readily be calculated using the program CompuSyn (CompuSyn, Paramus, N.J.). Chou himself (Chou 2006) defines an interaction as slightly synergistic if the CI value is 0.85-0.9, moderately synergistic if the CI value is 0.7-0.85, synergistic if the CI value is 0.3-0.7, strongly synergistic if the CI value is 0.1-0.3, and very strongly synergistic if the CI value is <0.1. In cancer therapy literature, the values of CI that define synergism can vary, for example in Lin et al., 2007, Carcinogenesis 28: 2521-2529, synergism between drugs was defined as CI<1, and in Fischel et al., 2006, Preclinical Report 17: 807-813, synergism was defined as CI<0.8. Similar numbers are used in the anti-viral therapy field. For example, in Wyles et al., 2008, Antimicrob Agents Chemotherapy 52: 1862-1864, synergism was defined as CI<0.9 and in Gantlett et al., 2007, Antiviral Res 75:188-197, synergism was defined as CI<0.9. Based on these references, synergism can be defined as CI values of <1.9. As shown in Example 2, the Chou-Talalay as well as the highest single agent (HSA), Bliss and Loewe models computed by the Combenefit software show a synergism of the combination of PD-0184264 and Baloxavir marboxil. Highest single agent (HSA), Bliss and Loewe models are, e.g., explained and reviewed in Foucquier and Guedj 2015 (Pharmacology Research & Perspectives 3(3):e00149).

The MEK inhibitor and the CEN inhibitor of the invention may have a synergistic effect in the treatment of a viral disease greater than the additive effect of each of the MEK inhibitor and the CEN inhibitor administered separately or in combination as predicted by a simple additive effect of the two drugs. In such a case, the synergistically effective amount of the MEK inhibitor is less than the amount needed to treat the viral infection if the MEK inhibitor was administered without the CEN inhibitor. Similarly, the synergistically effective amount of the CEN inhibitor is less than the amount needed to treat the viral infection or if the CEN inhibitor was administered without the MEK inhibitor. The synergistic amount of the MEK inhibitor and of the CEN inhibitor may be defined by the synergism factor (CI value). If defined by the synergism factor (CI value) than CI is less than about 0.9, alternatively less than about 0.85, alternatively less than about 0.8, alternatively less than about 0.75, alternatively less than about 0.7, alternatively less than about 0.65, alternatively less than about 0.6, alternatively less than about 0.55, alternatively less than about 0.5, alternatively less than about 0.45, alternatively less than about 0.4, alternatively less than about 0.35, alternatively less than about 0.3, alternatively less than about 0.25, alternatively less than about 0.2, alternatively less than about 0.15, alternatively less than about 0.1.

The combined use of a MEK inhibitor and a CEN inhibitor according to the invention provides a beneficial therapeutic effect also in case of viral disease wherein the virus or virus strain shows or has developed a resistance, in particular a resistance to a CEN inhibitor. In addition, the combined used may act to preserve the efficacy of both drugs over time because the development of resistance would not be observed at all or would be delayed in the time.

Baloxavir marboxil as CEN inhibitor may be used in combination with CI-1040 as MEK inhibitor in the method and/or pharmaceutical composition of the invention. Baloxavir marboxil as CEN inhibitor may be used in combination with PD-0184264 as MEK inhibitor in the use in the treatment and/or pharmaceutical composition of the invention. Baloxavir marboxil as CEN inhibitor may be used in combination with GSK-1120212 as MEK inhibitor in the use in the treatment and/or pharmaceutical composition of the invention. Baloxavir marboxil as CEN inhibitor may be used in combination with GDC-0973 as MEK inhibitor in the use in the treatment and/or pharmaceutical composition of the invention. Baloxavir marboxil as CEN inhibitor may be used in combination with PLX-4032 as MEK inhibitor in the use in the treatment and/or pharmaceutical composition of the invention. Baloxavir marboxil as CEN inhibitor may be used in combination with AZD6244 as MEK inhibitor in the use in the treatment and/or pharmaceutical composition of the invention. Baloxavir marboxil as CEN inhibitor may be used in combination with AZD8330 as MEK inhibitor in the use in the treatment and/or pharmaceutical composition of the invention. Baloxavir marboxil as CEN inhibitor may be used in combination with AS-703026 as MEK inhibitor in the use in the treatment and/or pharmaceutical composition of the invention. Baloxavir marboxil as CEN inhibitor may be used in combination with RDEA-119 as MEK inhibitor in the use in the treatment and/or pharmaceutical composition of the invention. Baloxavir marboxil as CEN inhibitor may be used in combination with RO-5126766 as MEK inhibitor in the use in the treatment and/or pharmaceutical composition of the invention. Baloxavir marboxil as CEN inhibitor may be used in combination with RO-4987655 as MEK inhibitor in the use in the treatment and/or pharmaceutical composition of the invention. Baloxavir marboxil as CEN inhibitor may be used in combination with PD-0325901 as MEK inhibitor in the use in the treatment and/or pharmaceutical composition of the invention. Baloxavir marboxil as CEN inhibitor may be used in combination with TAK-733 as MEK inhibitor in the use in the treatment and/or pharmaceutical composition of the invention. Baloxavir marboxil as CEN inhibitor may be used in combination with AS703026 as MEK inhibitor in the use in the treatment and/or pharmaceutical composition of the invention. Baloxavir marboxil as CEN inhibitor may be used in combination with PD98059 as MEK inhibitor in the use in the treatment and/or pharmaceutical composition of the invention. Baloxavir marboxil as CEN inhibitor may be used in combination with PD184352 as MEK inhibitor in the use in the treatment and/or pharmaceutical composition of the invention. Preferably, Baloxavir marboxil is combined with PD-0184264 (ATR-002) in the use in the treatment of the invention and the pharmaceutical composition of the invention.

In the use of the invention, a MEK inhibitor and a CEN inhibitor may be administered contemporaneously, previously or subsequently. The MEK inhibitor and a CEN inhibitor preferably are administered contemporaneously. They may be administered as a single formulation or in separate formulations. A single formulation is also described herein as the pharmaceutical composition of the invention.

The viral infection to be prevented or be treated by the combined administration of a MEK inhibitor and a CEN inhibitor of the invention is preferably an infection caused by negative RNA strand virus. More preferably, the viral disease is caused by an influenza virus, even more preferably the viral disease is caused by influenza A or B virus. Influenza viruses are for example: H1N1, H5N1, H7N7, and H7N9. In some cases, the viruses have developed resistance against an antiviral agent, such as a CEN inhibitor. Particularly preferred are the influenza A virus subtypes H1N1, H2N2, H3N2, H5N6, H5N8, H6N1, H7N2, H7N7, H7N9, H9N2, H10N7, N10N8 and/or H5N1.

In the use in the treatment of the invention or the use of the pharmaceutical composition wherein the MEK inhibitor and the CEN inhibitor are used in combination, the patient preferably is a mammal or a bird. Examples of suitable mammals include, but are not limited to, a mouse, a rat, a cow, a goat, a sheep, a pig, a dog, a cat, a horse, a guinea pig, a canine, a hamster, a mink, a seal, a whale, a camel, a chimpanzee, a rhesus monkey and a human. Examples of suitable birds include, but are not limited to, a turkey, a chicken, a goose, a duck, a teal, a mallard, a starling, a Northern pintail, a gull, a swan, a Guinea fowl or water fowl to name a few. Human patient are a particular embodiment of the present invention. A human patient is a particular embodiment of the present invention. The terms patient and subject are used interchangeably.

The MEK inhibitor may be administered orally, intravenously, intrapleurally, intramuscularly, topically or via inhalation. Preferably, the MEK inhibitor is administered via inhalation or orally.

The CEN inhibitor may be administered orally, intravenously, intrapleurally, intramuscularly, topically or via inhalation. Preferably, the CEN inhibitor is administered via inhalation or orally.

When the MEK inhibitor and the CEN inhibitor are in a single formulation such as in the pharmaceutical composition of the invention, the formulation may be administered orally, intravenously, intrapleurally, intramuscularly, topically or via inhalation. Preferably, the formulation is administered orally or via inhalation.

The use in the treatment of the invention may comprise treating a patient in need of treatment with a therapeutically effective amount of a MEK inhibitor or a pharmaceutically acceptable salt thereof; and simultaneously or sequentially a CEN inhibitor as described herein.

In one aspect, a method of treating a viral infection in a patient is provided comprising (1) administering to a patient in need of treatment a therapeutically effective amount of a compound which is a MEK inhibitor or a metabolite thereof or a pharmaceutically acceptable salt thereof; and simultaneously or sequentially (2) administering to said patient a therapeutically effective amount of Baloxavir marboxil or a pharmaceutically acceptable salt thereof. To put it differently, in accordance with this aspect, the method comprises administering a therapeutically effective amount of a MEK inhibitor or a metabolite thereof or a pharmaceutically acceptable salt thereof to a patient who is under treatment of Baloxavir marboxil or a pharmaceutically acceptable salt thereof or administering a therapeutically effective amount of Baloxavir marboxil or a pharmaceutically acceptable salt thereof to a patient who is under treatment with a MEK inhibitor or a metabolite thereof or a pharmaceutically acceptable salt thereof.

In one embodiment of the use in the treatment of the present invention, the compound MEK inhibitor can be administered orally or via inhalation at an effective therapeutic dosage, while the CEN inhibitor can be administered at a dose and dosing schedule as provided in the approved prescribing information or less, preferably at a lower dose (due to the synergistic effect). For example, according to Baloxavir marboxil label, Baloxavir marboxil is administered in capsules of 40 mg (40 to 80 kg subject weight) or 80 mg (more than 80 kg subject weight). A dosage of 40 mg or 80 mg as a single dose is the adults and adolescents standard dosage. A lower dosage may be used when Baloxavir marboxil is administered in combination with a MEK inhibitor. In one embodiment, the therapeutically effective amount of the MEK inhibitor is, e.g., from 0.1 mg to 2000 mg, 0.1 mg to 1000 mg, 0.1 to 500 mg, 0.1 to 200 mg, 30 to 300 mg, 0.1 to 75 mg, 0.1 to 30 mg.

In the sequential combination therapies discussed herein, preferably the drugs in sequential combination are administered according to their pharmacokinetic profiles such that the second drug is administered after the plasma level of the first drug is substantially reduced or removed. The pharmacokinetic profiles of the MEK inhibitor and the CEN inhibitor drugs are generally known in the art.

As outlined above, the present invention further provides a pharmaceutical composition comprising a MEK inhibitor or a pharmaceutically acceptable salt or metabolite thereof and a cap-dependent endonuclease inhibitor for use as a medicament. In one specific embodiment, the pharmaceutical composition of the invention is for use in the prophylaxis and/or treatment of a viral infection, preferably an infection caused by a negative RNA strand virus, more preferably by an influenza virus and most preferably by an influenza A or influenza B virus.

The pharmaceutical composition of the invention may be in the form of orally administrable suspensions or tablets; nasal sprays, sterile injectable preparations (intravenously, intrapleurally, intramuscularly), for example, as sterile injectable aqueous or oleaginous suspensions or suppositories. When administered orally as a suspension, these compositions are prepared according to techniques available in the art of pharmaceutical formulation and may contain microcrystalline cellulose for imparting bulk, alginic acid or sodium alginate as a suspending agent, methylcellulose as a viscosity enhancer, and sweeteners/flavoring agents known in the art. As immediate release tablets, these compositions may contain microcrystalline cellulose, di-calcium phosphate, starch, magnesium stearate and lactose and/or other excipients, binders, extenders, disintegrants, diluents, and lubricants known in the art. The injectable solutions or suspensions may be formulated according to known art, using suitable non-toxic, parenterally acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid. The pharmaceutical compounds in the method of present invention can be administered in any suitable unit dosage forms. Suitable oral formulations also in context of the pharmaceutical composition of the invention can be in the form of tablets, capsules, suspension, syrup, chewing gum, wafer, elixir, and the like. Pharmaceutically acceptable carriers such as binders, excipients, lubricants, and sweetening or flavoring agents can be included in the oral pharmaceutical compositions. If desired, conventional agents for modifying tastes, colors, and shapes of the special forms can also be included.

For injectable formulations, the pharmaceutical compositions can be in lyophilized powder in admixture with suitable excipients in a suitable vial or tube. Before use in the clinic, the drugs may be reconstituted by dissolving the lyophilized powder in a suitable solvent system to form a composition suitable for intravenous or intramuscular injection.

In one embodiment, the pharmaceutical composition can be in an orally administrable form (e.g., tablet or capsule or syrup etc.) with a therapeutically effective amount (e.g., from 0.1 mg to 2000 mg, 0.1 mg to 1000 mg, 0.1 to 500 mg, 0.1 to 200 mg, 30 to 300 mg, 0.1 to 75 mg, 0.1 to 30 mg) of MEK inhibitor and a therapeutically effective amount of CEN inhibitor as described above. For example, according to Baloxavir marboxil label, Baloxavir marboxil is administered in capsules of 40 mg (40 to 80 kg subject weight) or 80 mg (more than 80 kg subject weight). A dosage of 40 mg or 80 mg as a single dose is the adults and adolescents standard dosage. A lower dosage may be used when Baloxavir marboxil is administered in combination with a MEK inhibitor.

The therapeutically effective amount for each active compound can vary with factors including but not limited to the activity of the compound used, stability of the active compound in the patient's body, the severity of the conditions to be alleviated, the total weight of the patient treated, the route of administration, the ease of absorption, distribution, and excretion of the active compound by the body, the age and sensitivity of the patient to be treated, adverse events, and the like, as will be apparent to a skilled artisan. The amount of administration can be adjusted as the various factors change over time.

In accordance with another aspect of the present invention, a pharmaceutical kit is provided comprising, in a compartmentalized container, (1) a unit dosage form of a MEK inhibitor such as PD-0184264, PLX-4032, AZD6244, AZD8330, AS-703026, GSK-1120212, RDEA-119, RO-5126766, RO-4987655, 01-1040, PD-0325901, GDC-0973, TAK-733, PD98059 and PD184352 and (2) a unit dosage form of a CEN inhibitor such Baloxavir. Optionally, the kit further comprises instructions for using the kit in the combination therapy method in accordance with the present invention.

### Definitions

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms.

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or” as used herein.

### EXAMPLES

### Example 1: Comparison Between MEK Inhibitors and Other Standard of Care

Reagents

A549 cells (ATCC® CCL-185™), 0.3% triton-x-100, MDCK II cells (ATCC® CRL-2936™), 0.1% tween 20, Phosphate-buffered saline (PBS, Gibco Cat. No.: 14190144), PBS+10% FCS+0.1% tween 20, Infection PBS, Roti®-Histofix 10% (Roth, Cat. No.: A146.1)→Prepare working solution 4%, TPCK-trypsin, Primary antibody (anti-NP; AA5H, Cat. No.: MCA400), 2×MEM, Secondary antibody (peroxidase-labeled anti-mouse antibody, Cat. No.: 115-035-003), Albumine fraction V solution, KPL True Blue™ (Cat. No.: 5510-0049), Avicel 2.5% (RC-581, FMC BioPolymer).

Method

Day 1

1—Plating two 24-well plates
a. Cell type: A549
b. Seeding density: 0.5×105 cell/ml

2—Incubate for 24 h

Day 2

3—Check the confluency of the prepared 24-well plates

4—Remove media and wash 2× with PBS

Virus Dilution

5—Perform tenfold serial dilution of the virus (titer: 6.0×107 pfu/ml)

6—Inoculate each well with 0.001 MOI

7—Incubate for 45 min

Preparation of Concentrations of the Tested Substance

8—Add TPCK-trypsin at final conc. 2 μg/ml to infection media

ATR002
Tested compound: ATR002
Solvent: DMSO
Concentration: Stock solution 10 mM, working solution: 1 mM
Prepare the following concentrations: 50, 10, 2, and 0.4 μM in infection medium

Baloxavir Marboxil
Tested compound: Baloxavir Marboxil (BLXM)
Concentration: stock solution 1 mM solvent: DMSO
Working solution: 100 nM
Prepare the following concentrations: 1, 0.2, 0.04 and 0.008 nM in infection medium

9—Prepare combinations in a 4×4 matrix

10—Prepare DMSO control at final conc. 1% in infection medium

The 24-Well Plate and Test Substance

11—Check confluency of the plate after incubation

12—Remove the inocula

13—Add 1 ml of each conc. to each well

14—Incubate for 22 h

Preparing 96-Well Plates

15—Prepare thirteen 96-well plates
a. Cell type: MDCK II
b. Seeding density: 3×10⁵ cell/well

16—Incubate for 24 h

Day 3

The 24-Well Plates and Tested Substances

17—Make two aliquots of each conc. in Eppendorf 1.5 ml, 300 μl in each tube. Store one in −80 C

Preparing 96-Well Plates (U-Shape)

18—Prepare the same number of previously prepared 96-well plates by adding 100 μl infection PBS in each well of U-shape

19—Add to the first well of each column 50 μl of its corresponding conc.
Each plate has two columns corresponding to −ve and +ve controls

20—After adding conc. to each first well, make serial dilution by moving 50 μl form the first well to the following one. At the end, discard the last 50 μl

The MDCK II 96-Well Plates

21—Check confluency

22—Remove the growth media and wash 2× with PBS

23—Transfer the dilutions prepared in U-shape 96-well plates to MDCK II plates

24—Incubate for 1 h

Preparation of the Avicel Overlay

25—Mix 1:1 2×MEM media and 2× Avicel

26—Add TPCK-trypsin at final conc. 2 μg/ml

27—After the incubation period, discard the inocula, and apply 100 μl/well of the Avicel overlay

28—Incubate for 22 h

Day 4

Fixation and Staining

29—After 22 h, fix with 4% paraformaldehyde solution for 30 min at 4° C. and washed 2× with PBS

30—Add 100 μl/well 0.3% triton-x-100 prepared in PBS and Incubate 10 min

31—Discard it then add 100 μl/well 10% FCS (fresh prepared in PBS)

32—Incubate on shaker for 10 min

33—Discard it then add 50 μl primary antibody (anti-NP; AA5H)

34—Incubate 60 min on shaker

35—Wash (3×) for 5 min with (PBS+0.1% tween 20)

36—Add 50 μl secondary antibody (peroxidase-labeled anti-mouse antibody)

37—Incubate 30-60 min on shaker

38—Wash (3×) for 5 min with (PBS+0.1% tween 20)

39—Add 50 μl True Blue™ for 10 min

40—Wash with water then let it to dry

41—Perform data analysis

Results

As depicted in FIG. 1, both oseltamivir and CI-1040 are very effective against wild type (wt) strain of A/Mississippi/3/2001 (H1N1). In contrast, while demonstrating the antiviral potential of both drugs against the mutant strain with the H275Y mutation in the NA gene, significant reduction in oseltamivir effectiveness was observed while CI-1040 showed a comparable antiviral effect which quite similar as the wild strain.

To further evaluate the potential antiviral activity of ATR002 (the active metabolite of CI-1040), the inventors compared the antiviral activity of ATR002 versus the newly licensed anti-influenza virus drug Baloxavir marboxil (BLXM) which designed to inhibit the cap-dependent endonuclease protein. As shown in FIG. 2A, BLXM was very potent against the wild type rgA/Giessen/6/09 (H1N1-WT) with an approximate complete reduction of the viral titer while ATR002 activity was lower by 13%. Conversely, the BLXM activity was lower by 37% when investigated using the mutant strain rgA/Giessen/6/09 (H1N1)-PA-I38T but ATR002 showed steady effect as found in the wild type. Likewise, while demonstrating the antiviral activity using rgA/Victoria/3/75 (H3N2-WT) and rgA/Victoria/3/75 (H3N2-PA-I38T) (FIG. 2B), ATR002 revealed its potency against both variants, whereas, BLXM lost about 41% of its activity.

### Example 2: Synergistic Effect Between ATR002 and Baloxavir Marboxil

Material and Methods

Drugs

The MEK inhibitor ATR-002 (PD0184264) [2-(2-chloro-4-iodophenylamino)-N-3,4-difluoro benzoic acid, the active metabolite of CI-1040, was synthesized at ChemCon GmbH (Freiburg, Germany).

Baloxavir marboxil, the cap-dependent endonuclease of influenza virus, was purchased from Hycultec GmbH (Cat: HY-109025) and prepared for a working solution 1 mM according to the manufacturer instructions.

Cells and Viruses

Human lung adenocarcinoma cells (A549, ATCC® CCL185™) and Madin-Darby canine kidney cells (MDCK II, ATCC® CRL2936™) were purchased from ATCC and cultured in Iscove's Modified Dulbecco's Medium (IMDM) supplemented with 10% FBS and 100 U/ml Penicillin-Streptomycin.

Influenza virus H1N1 was used in the virus inhibition experiments with 0.001 MOI

Virus Inhibition Assay

The susceptibility of influenza virus to ATR-002 or other drugs such as Baloxavir marboxil was determined by measuring the reduction in FFU in the presence of the drugs. Different concentrations (0.4-50 μM) of ATR-002 and (0.008-1 nM) Baloxavir marboxil were prepared by making 5-fold serial dilution in influenza virus infection medium (DMEM media supplemented with 0.2% BSA, 1 mM MgCl₂, 0.5 mM CaCl₂, 100 U/mL penicillin, 0.1 mg/mL streptomycin, and 2 μg/ml TPCK-treated Trypsin) supplemented with 1 μg/ml L-tosylamido 2-phenylethyl chloromethyl ketone (TPCK)-treated trypsin. A549 cells (Human lung adenocarcinoma cell line (A549, ATCC® CCL185™) was purchased from ATCC and cultured in Iscove's Modified Dulbecco's Medium (IMDM) supplemented with 10% FBS and 100 U/mL Penicillin-Streptomycin). Cells were kept in a 37° C. and 5% CO₂ atmosphere and were infected with H1N1 in 24-well plate and incubated for 45 min. After incubation, the inocula were removed, the confluent monolayers washed with PBS and supplemented with infection medium containing the tested drugs. The cell culture supernatant corresponding to each treatment was collected after 24 h and subjected to focus reduction assay using MDCK 11 (Madin-Darby canine kidney cells (MDCK 11, ATCC® CRL2936™) were purchased from ATCC and cultured in Iscove's Modified Dulbecco's Medium (IMDM) supplemented with 10% FBS and 100 U/mL Penicillin-Streptomycin. Cells were kept in a 37° C. and 5% CO₂ atmosphere) as previously described (Matrosovich et al., 2006, Virol J. 31(3):63).

Analysis of Synergy/Antagonism from Combination Studies

In order to determine the possible additive and synergistic effects when using combinations of PD0184264 with Baloxavir marboxil, the data from virus inhibition assay were first analyzed using the Combenefit software (Di Veroli et al., 2016, Bioinformatics 32(18):2866-2868), which simultaneously assesses synergy/antagonism using three published models (Highest single agent (HSA), Bliss, and Loewe).

Dose-response curves were also included for each individual compound to generate a dose-response surface for the reference models, from which the experimental surface and modelled surface were then compared. At each combination, deviations in the experimental surface from the modelled surface were attributed a percentage score indicating the degree of either synergy (increased effect) or antagonism (decreased effect). The “Contour” and “surface” plots were selected as graphical outputs for the synergy distribution.

Data were also analyzed according to the Chou-Talalay model using CompuSyn software (Chou, 2010, Cancer Res 70(2):440-446). The software calculates the combination index (CI) for each drug combination, where a CI value <1 indicates synergy, CI=1 is additive and CI>1 indicates antagonism.

Results

Influenza viruses (IV) infection is a public health concern worldwide. Currently, all available vaccines as well as antiviral drugs that target the virus itself are prone to resistance. It is proven that influenza viruses able to modulate and control cellular pathways involved in the viral life cycle like Raf/MEK/ERK signal pathway which the nuclear export of vRNPs is strongly dependent on the virus-induced activation. Along this line, the inventors demonstrated earlier the antiviral potential of MEK inhibitor PD0184264 (ATR002), the active metabolite of CI-1040 against influenza viruses over in vitro and in vivo levels (Example 1, see also WO 2019/076947). Recently, a newly licensed antiviral drug so-called Baloxavir marboxil (Xofluza), which was designed to inhibit the cap-dependent endonuclease protein, has demonstrated efficacy in a wide range of influenza viruses, including oseltamivir-resistant strains. However, the emergence of resistant variants against the newly licensed drug has already been reported.

Given to both the recently licensed anti-influenza drug Baloxavir marboxil and the potential MEK inhibitor (ATR002) as a therapeutic option, the inventors investigated whether the combination between these two drugs would augment the antiviral activity. Surprisingly, there is an increase in the antiviral activity at different concentrations of ATR002 (0.4, 2, and 10 μM) when combined with BLXM (0.008 and 0.04 nM) indicated by the reduction in viral titer compared to the individual treatment of each drug. Moreover, it can be inferred form Chou-Talalay model that the combination at lower concentrations of ATR002 and BLXM leads to a strong synergistic effect with low CI values (see FIG. 4A and Table 1). Table 2 and FIG. 4B also show a strong synergistic effect as expressed in the drug dose reduction index (DRI). These data were in agreement with the most widely used models (HAS, Bliss, and Loewe) which also revealed that the combinations at higher doses lead stronger additive effect rather than synergistic effect (FIG. 3).

TABLE 1
Combination Index (CI) values for drug combos

| Conc. BLXM | Conc. ATR002 | |
|---|---|---|
| (nM) | (μM) | CI |
| 0.008 | 0.4 | 0.17469 |
| 1 | 10 | 0.24757 |
| 0.008 | 10 | 0.28142 |
| 1 | 50 | 0.29305 |
| 0.2 | 50 | 0.35104 |
| 0.008 | 50 | 0.42303 |
| 0.008 | 2 | 0.44177 |
| 0.2 | 10 | 0.63435 |
| 0.04 | 2 | 0.91172 |
| 0.04 | 0.4 | 1.18204 |
| 1 | 2 | 1.31281 |
| 0.2 | 2 | 1.62481 |
| 1 | 0.4 | 1.94132 |
| 0.2 | 0.4 | 2.31597 |
| 0.04 | 10 | 2.92652 |
| 0.04 | 50 | 3.32808 |

TABLE 2
Drug Dose Reduction (DRI) data example
of BLXM and ATR002 predicted combos

| | Dose BLXM | Dose^{a} ATR002 | DRI | DRI |
|---|---|---|---|---|
| Fa ^{b} | (nM) | (μM) | BLXM | ATR002 |
| 0.99 | 4.23358^{a} | 879.684^{a} | 4.23358 | 17.5937 |
| 0.97 | 1.50936^{a} | 228.8^{a} | 7.54681 | 4.57599 |
| 0.95 | 0.92466 | 120.664^{a} | 115.583 | 2.41327 |
| 0.9 | 0.4644 | 49.0947 | 2.32201 | 4.90947 |
| 0.88 | 0.38452 | 38.3704 | 48.0652 | 3.83704 |
| 0.6 | 0.08906 | 5.68262 | 11.133 | 2.84131 |
| 0.58 | 0.08253 | 5.14432 | 10.316 | 12.8608 |
| 0.57 | 0.07947 | 4.89711 | 1.98684 | 2.44855 |

^{a}predicted dose that shift from its empirical estimation
^{b} fraction of uninfected infected cells or inhibitory effect

1. MEK inhibitor for the use in the treatment or prevention of a viral infection in combination with a cap-dependent endonuclease inhibitor.

2. The MEK inhibitor for the use of claim 1, wherein the MEK inhibitor is selected from the group consisting of CI-1040, PD-0184264 GSK-1120212, GDC-0973, PLX-4032, AZD6244, AZD8330, AS-703026, RDEA-119, RO-5126766, RO-4987655, PD-0325901, TAK-733, AS703026, PD98059 and PD184352 or pharmaceutically acceptable salt or metabolite thereof.

3. The MEK inhibitor for the use of claim 1 or 2, wherein the cap-dependent endonuclease inhibitor is Baloxavir marboxil.

4. The MEK inhibitor for the use of claim 3, wherein the MEK inhibitor is CI-1040 or PD-0184264.

5. The MEK inhibitor for the use of any one of claims 1 to 4, wherein the viral infection is an infection caused by a negative RNA strand virus.

6. The MEK inhibitor for the use of to claim 5, wherein the virus is influenza virus.

7. The MEK inhibitor for the use of claim 5, wherein the influenza virus is influenza A virus or influenza B virus.

8. The MEK inhibitor for the use of any one of claims 1 to 7, wherein the MEK inhibitor is administered contemporaneously, previously or subsequently to the cap-dependent endonuclease inhibitor.

9. A pharmaceutical composition comprising a MEK inhibitor or a pharmaceutically acceptable salt or metabolite thereof and a cap-dependent endonuclease inhibitor for use as a medicament.

10. The pharmaceutical composition for the use of claim 9 wherein the MEK inhibitor is selected from CI-1040, PD-0184264, GSK-1120212, GDC-0973, PLX-4032, AZD6244, AZD8330, AS-703026, RDEA-119, RO-5126766, R04987655, PD-0325901, TAK-733, AS703026, PD98059 and PD184352, or a pharmaceutically acceptable salt or metabolite thereof.

11. The pharmaceutical composition for the use of claim 9 or 10 wherein the cap-dependent endonuclease inhibitor is Baloxavir marboxil.

12. The pharmaceutical composition for the use of claim 11, wherein the MEK inhibitor is CI-1040 or PD-0184264.

13. The pharmaceutical composition as defined in any one of claims 9 to 12 for the use in the prophylaxis and/or treatment of a viral infection.

14. The pharmaceutical composition for the use claim 13 wherein the viral infection is an infection caused by a negative RNA strand virus.

15. The pharmaceutical composition for the use claim 14, wherein the virus is influenza virus, preferably an influenza A virus or influenza B virus.
